(19) United States
(12) Patent Application Publication
(10) Pub. No.: US 2021/0219858 A1
(43) Pub. Date: Jul. 22, 2021

(54) UNIVERSAL COGNITIVE STATE DECODER BASED ON BRAIN SIGNAL AND METHOD AND APPARATUS FOR PREDICTING ULTRA-HIGH PERFORMANCE COMPLEX BEHAVIOR USING THE SAME

(71) Applicant: KOREA ADVANCED INSTITUTE OF SCIENCE AND TECHNOLOGY, Daejeon (KR)

(72) Inventors: Sang Wan Lee, Daejeon (KR); Dongjae Kim, Daejeon (KR)

(73) Assignee: KOREA ADVANCED INSTITUTE OF SCIENCE AND TECHNOLOGY, Daejeon (KR)

(21) Appl. No.: 16/950,095

(22) Filed: Nov. 17, 2020

(30) Foreign Application Priority Data
Jan. 16, 2020 (KR) 10-2020-0005994

(51) Int. Cl. A61B 5/04; A61B 5/00; A61B 5/0476; A61B 5/16; G16H 20/70; G06N 3/08; G06N 3/04

(52) U.S. Cl. CPC A61B 5/04012; A61B 5/7267; A61B 5/0476; G06N 3/04; G16H 20/70; G06N 3/08; A61B 5/165

(57) ABSTRACT

Disclosed are a universal cognitive state decoder based on a brain signal and a method and apparatus for predicting an ultra-high performance complex behavior using the same. The method of predicting a complex behavior may include configuring a high-level cognitive state decoder based on a brain signal for classifying a human's high-level core cognitive state, configuring a universal cognitive state decoder by including a calculated value of the high-level cognitive state decoder in another cognitive state decoder as an input value, and predicting a human's complex behavior using the universal cognitive state decoder.

### CROSS REFERENCE TO RELATED APPLICATION

This application claims priority under 35 U.S.C. § 119 to Korean Patent Application No. 10-2020-0005994 filed on Jan. 16, 2020, which is incorporated herein by reference in its entirety.

### BACKGROUND OF THE INVENTION

### 1. Technical Field

The following embodiments relate to a universal cognitive state decoder based on a brain signal and a method and apparatus for predicting an ultra-high performance complex behavior using the same.

### 2. Description of the Related Art

The existing engineering application systems based on a brain signal have been developed for the purpose of rehabilitating an exercise function of a paraplegic patient, for example. However, such a system has the following limits. The engineering application system based on a brain signal is based on an active or conscious generation signal. Accordingly, the engineering application system needs to generate an active exercise function signal (e.g., motor intention) from the brain through a concentration, and accumulates excessive fatigue in a user in the concentration process, thus having a low wide use. Furthermore, the engineering application system based on a brain signal has limited availability due to low spatial resolution of an exercise function signal. In the rehabilitation of an exercise function based on brainwaves, only simple exercise functions can be classified (e.g., distinguish between movements of the left arm and the right arm) due to low spatial resolution of a signal, but a human's complicated behavior and decision making cannot be predicted. Furthermore, the engineering application system based on a brain signal cannot be widely used. One system cannot be used to classify a different type of an exercise function because a brainwave signal has a quite different characteristic depending on the type of exercise or task.

However, such a limit of the engineering application system based on a brain signal is not applied to a “cognitive state”, that is, a function of the brain having a much higher dimension than a task or exercise level. Such a high-level cognitive state not dependent on a task has the following advantages.

A high-level cognitive state is a natural and unconscious generation signal. Unlike an exercise function in which an active signal needs to be transmitted in order to adjust muscle, the high-level cognitive state is an advanced function performed in the brain involuntarily and unconsciously. Accordingly, the high-level cognitive state has a small issue, such as noise, because fatigue is not accumulated in a user. Furthermore, the high-level cognitive state has high availability in a high-dimensional brain function. A human's complicated behavior can be specifically predicted by directly reading a cognitive function because a complicated behavior pattern exceeding a simple exercise function is adjusted by a high-dimensional cognitive function, such as a strategy, inference, a plan or an emotion. Furthermore, the high-level cognitive state has a universal signal characteristic. Although the type of cognitive function is determined based on a language, there are many types of similar cognitive functions which are not fully classified on a brain signal and context basis. Accordingly, a system for classifying specific cognitive states (e.g., fatigue, stress, sleepiness, non-vigilance, and anxiety) may be used to classify another type of cognitive state.

Korean Patent No. 10-1285821 relates to a measurement method of cognitive fatigue and an apparatus adopting the method, and describes a technology capable of evaluating fatigue from a cognitive viewpoint including a low-dimensional cognitive response and a high-dimensional cognition response.

### BRIEF SUMMARY OF THE INVENTION

Embodiments describe a universal cognitive state decoder based on a brain signal and a method and apparatus for predicting an ultra-high performance complex behavior using the same, and more specifically, provide a universal cognitive state decoding technology having a neural signal, such as brainwaves, as input based on a functional characteristic of a cognitive state and a technology for implementing a complicated behavior prediction system using the technology.

Furthermore, embodiments provide a universal cognitive state decoder using a high-level cognitive state decoder based on a brain signal for identifying a human's high-level core cognitive state, and provide a universal cognitive state decoder based on a brain signal, which can predict a human's complicated behavior pattern and a method and apparatus for predicting an ultra-high performance complex behavior using the same.

In an aspect, a method of predicting a complex behavior may include configuring a high-level cognitive state decoder based on a brain signal for classifying a human's high-level core cognitive state, configuring a universal cognitive state decoder by including a calculated value of the high-level cognitive state decoder in another cognitive state decoder as an input value, and predicting a human's complex behavior using the universal cognitive state decoder.

The method may further include designing a Markov decision-making task for extracting a task-independent core cognitive state, before configuring the high-level cognitive state decoder.

Configuring the high-level cognitive state decoder based on the brain signal for classifying the human's high-level core cognitive state may include training the high-level cognitive state decoder using a goal-directed cognitive state and a habitual cognitive state, that is, task-independent core cognitive states.

Configuring the high-level cognitive state decoder based on the brain signal for classifying the human's high-level core cognitive state may include estimating a core cognitive state for a behavior strategy inherent in decision making of a human behavior based on a computational model derived from decision-making neuroscience research using the high-level cognitive state decoder.

Furthermore, configuring the high-level cognitive state decoder based on the brain signal for classifying the human's high-level core cognitive state may include estimating a core cognitive state for decision making by combining a behavior strategy inherent in the decision making of a human behavior with characteristics of a brain signal using the high-level cognitive state decoder.

Estimating the core cognitive state for decision making by combining the behavior strategy inherent in the decision making of the human behavior with characteristics of the brain signal using the high-level cognitive state decoder may include estimating a cognitive state for the decision making by classifying each decision-making strategy using a convolution neural network (CNN).

Furthermore, estimating the core cognitive state for decision making by combining the behavior strategy inherent in the decision making of the human behavior with characteristics of the brain signal using the high-level cognitive state decoder may include estimating a cognitive state for the decision making by visualizing characteristics of a brain signal associated with each decision-making strategy in a class activation map (CAM) form.

Configuring the universal cognitive state decoder by including the calculated value of the high-level cognitive state decoder in the another cognitive state decoder as the input value may include configuring the universal cognitive state decoder by including the calculated value of the high-level cognitive state decoder in a plurality of other cognitive state decoders as the input value.

In this case, the high-level cognitive state decoder and the universal cognitive state decoder may be convolution neural network (CNN)-based decoders.

Predicting the human's complex behavior using the universal cognitive state decoder may include predicting the complex behavior according to a reinforcement learning strategy using the universal cognitive state decoder, and inferring computer-recognizable behaviors according to vigilance and non-vigilance.

In another aspect, an apparatus for predicting a complex behavior may include a high-level cognitive state decoder based on a brain signal configured to classify a human's high-level core cognitive state, and a universal cognitive state decoder configured to predict a human's complex behavior by including a calculated value of the high-level cognitive state decoder in another cognitive state decoder as an input value.

The apparatus may further include a Markov decision-making task unit configured to design a Markov decision-making task for extracting a task-independent core cognitive state.

The high-level cognitive state decoder may train the high-level cognitive state decoder using a goal-directed cognitive state and a habitual cognitive state, that is, task-independent core cognitive states.

The high-level cognitive state decoder may include a behavior strategy prediction unit configured to estimate a core cognitive state for a behavior strategy inherent in decision making of a human behavior based on a computational model derived from decision-making neuroscience research using the high-level cognitive state decoder.

Furthermore, the high-level cognitive state decoder may include a decision-making prediction unit configured to estimate a core cognitive state for decision making by combining a behavior strategy inherent in the decision making of a human behavior with characteristics of a brain signal using the high-level cognitive state decoder.

The decision-making prediction unit may estimate a cognitive state for the decision making by classifying each decision-making strategy using a convolution neural network (CNN).

Furthermore, the decision-making prediction unit may estimate a cognitive state for the decision making by visualizing characteristics of a brain signal associated with each decision-making strategy in a class activation map (CAM) form.

The universal cognitive state decoder may be configured to include the calculated value of the high-level cognitive state decoder in a plurality of other cognitive state decoders as the input value.

In this case, the high-level cognitive state decoder and the universal cognitive state decoder may be convolution neural network (CNN)-based decoders.

The universal cognitive state decoder may predict the complex behavior according to a reinforcement learning strategy and infer computer-recognizable behaviors according to vigilance and non-vigilance.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a diagram schematically illustrating a conventional brain-computer interface (BCI) system.

FIG. 2 is a diagram schematically illustrating a BCI system according to an embodiment.

FIG. 3 is a diagram for describing the inference of a behavior strategy and decision making according to an embodiment.

FIG. 4 is a diagram for describing a BCI system according to an embodiment.

FIG. 5A is a diagram illustrating electroencephalogram (EEG) channel information of a 1-D convolution neural network (CNN) model according to an embodiment.

FIG. 5B is a diagram illustrating EEG channel information of a 2-D CNN model according to an embodiment.

FIG. 5C is a diagram illustrating EEG channel information of a 3-D CNN model according to an embodiment.

FIG. 6A is a diagram illustrating the structure of the 1-D CNN model according to an embodiment.

FIG. 6B is a diagram illustrating the structure of the 2-D CNN model according to an embodiment.

FIG. 6C is a diagram illustrating the structure of the 3-D CNN model according to an embodiment.

FIG. 7A is a diagram for describing a 2-D CNN class activation map (CAM) according to an embodiment.

FIG. 7B is a diagram illustrating simulation results of the 2-D CNN CAM according to an embodiment.

FIG. 8A is a diagram for describing a 3-D CNN CAM according to an embodiment.

FIG. 8B is a diagram illustrating simulation results of the 3-D CNN CAM according to an embodiment.

FIG. 9 is a diagram illustrating a complex behavior decoding concept diagram using a high-level cognitive state according to an embodiment.

FIG. 10 is a diagram for describing a high-level cognitive state decoder and the design of a behavior signal decoder using the same according to embodiments.

FIG. 11 illustrates a high-level cognitive state decoder and a behavior signal decoding method using the same according to embodiments.

FIG. 12A is a diagram illustrating performance of a decision-making strategy prediction decoder according to an embodiment.

FIG. 12B is a diagram illustrating performance of a behavior prediction decoder according to an embodiment.

FIG. 13 is a diagram for describing the design of a universal cognitive state decoder using a high-level cognitive state decoder according to an embodiment.

FIG. 14 is a diagram illustrating an apparatus for predicting an ultra-high performance complex behavior using the universal cognitive state decoder based on a brain signal according to an embodiment.

FIG. 15 is a flowchart illustrating a method of predicting an ultra-high performance complex behavior using the universal cognitive state decoder based on a brain signal according to an embodiment.

FIG. 16A is a diagram illustrating an example of a universal complex behavior decoder according to an embodiment.

FIG. 16B is a diagram illustrating performance evaluation of the universal cognitive state decoder according to an embodiment.

### DETAILED DESCRIPTION OF THE INVENTION

Hereinafter, embodiments are described in detail with reference to the accompanying drawings. However, the described embodiments may be modified in various other forms, and the scope of the present disclosure is not restricted by the following embodiments. Furthermore, various embodiments are provided to more fully describe the present disclosure to a person having average knowledge in the art. The shapes, sizes, etc. of elements in the drawings may be exaggerated for a clear description.

The following restrictions are imposed in producing a brain-computer interface (BCI) for detecting a human's decision-making strategy as a brain signal. The decision-making strategy is a high-dimensional process, and is inevitably detected as a self-report having relatively low reliability because it includes a human psychology. Furthermore, a human decision-making strategy inference BCI produced through a self-report does not have a neuroscience evidence of the decision-making strategy. Furthermore, a brain signal has a different characteristic depending on its type, and may be used to measure only some signals. Accordingly, a process of finding a proper brain signal needs to be previously performed in order to produce a BCI.

In order to solve such restrictions, an embodiment of the present disclosure proposes a new BCI system having a form in which psychology for a human's decision-making strategy and theories used in neuroscience research are actively used in the BCI.

Psychology and neuroscience research for a human's learning theoretically verify a decision-making strategy present in a human behavior baseline. As a result, a human's complicated decision-making process may be understood. In this case, each of research results has the following characteristic. Psychology research establishes a human's characteristic decision-making strategy and a behavioral characteristic thereof. Furthermore, the neuroscience research presents an evidence of a decision-making process within a human's brain through a theory of a human decision-making process researched through psychology research.

As a result, a proposed new BCI system is configured as follows. 1) Psychological prior-research of a human's decision making provides a theoretical base regarding how a decision-making strategy inherent in the human being affects a human's a behavior pattern. 2) Furthermore, neuroscience research of the human's decision making prepares a ground regarding in which area of the brain a corresponding decision-making strategy occurs, including whether the corresponding decision-making strategy is actually present in the brain. 3) A BCI system for inferring a human's decision-making strategy may be produced based on the theory applied to find the human's behavior and the brain signal according to the decision-making strategy in the previous two researches.

A proposed BCI system is trained through a computational model that directly infers a decision-making strategy. In particular, the proposed BCI system has characteristics in that decision-making strategies can be classified using a convolution neural network (CNN) and characteristics of a brain signal associated with each decision-making strategy can also be visualized in the form of a class activation map (CAM). Accordingly, a scientific ground on which the BCI system classifies decision-making strategies can also be analyzed.

Accordingly, the proposed BCI system may solve the aforementioned restrictions as follows. The proposed BCI system has high reliability because it deletes a self-report process and infers a cognitive state using a human's behavior pattern and a theory of a human's decision making detected in a brain signal. The theory used to infer the decision-making strategy naturally has a neuroscience ground because it is statistically relevant to a brain signal. Furthermore, a brain signal from which a decision-making strategy can be inferred may be determined through a brain area that participates in a decision-making process.

According to embodiments, an apparatus does not simply infer a human's inherent cognitive state, but infers a fundamental behavior strategy (e.g., learning strategy) of a human behavior using only brainwaves, and may infer a human's behavior and behavior strategy using brainwaves prior to the behavior. The apparatus is the same as a convention BCI system in that it infers a human's inherent state, but is different from the convention BCI system in that it infers even a behavior in a corresponding goal and a behavior strategy, that is, a source for the behavior. In particular, the apparatus is special in terms of a learning process composed of a combination of various brain areas and an interaction therebetween not a specific cognitive “state”, such as an emotion, and the inference of a strategy thereof. Many BCI technology development cases for reading decision making are present, but any previous technology development case for reading a behavior strategy is not present.

The following embodiments relate to a BCI system of a new concept for simultaneously estimating a “behavior strategy”, included in a human's decision making, and “decision making.” A “behavior strategy” may be estimated using a computational model derived from decision-making neuroscience research. Furthermore, “decision making” may be estimated by combining the estimated “behavior strategy” with characteristics of a brain signal.

The conventional BCI system has an object of classifying external signals which may be clearly identified like a human's exercise function. In contrast, an embodiment of the present disclosure aims to read and use an internal signal, such as a “behavior strategy” and “decision making” inherent in a “behavior.”

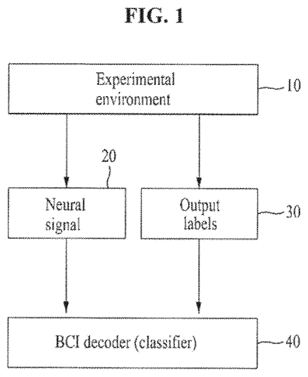

FIG. 1 is a diagram schematically illustrating a conventional brain-computer interface (BCI) system.

Referring to FIG. 1, in the conventional BCI system, a BCI decoder 40 is trained by a neural signal 20 and a usage-defined class output level 30 extracted from an experimental environment 10.

The existing BCI has usefulness in that it aims to read an exercise function (e.g., right arm/left arm), but in general, has strong intensity of an electroencephalogram (EEG) signal related to an exercise function and a label value that externally appears clearly. For example, the existing BCI adopts a method of labeling each EEG data as a movement of the right arm/left arm after EEG measurement when the right arm/left arm moves and training the BCI decoder using the EEG data.

However, such classification requires another approach method for a BCI for a cognitive state, which does not clearly appear. Representative research of the classification of cognitive states through EEG includes “emotion classification” and “fatigue state classification.” In these researches, however, an experimenter determines the label of each EEG data regardless of a brain signal. That is, there is no classification criterion for a cognitive state that does not appear to the eye.

In this aspect, neuroscience research provides a criterion for better classifying cognitive state EEG data. This is a computational model. In common neuroscience research, a computational model for each cognitive state is generated, and functional magnetic resonance imaging (fMRI) data is analyzed. Likewise, this is also inherent. In an embodiment of the present disclosure, a proper criterion for classifying cognitive state EEG data may be established using the results of fMRI neuroscience research and a computational model used for such research.

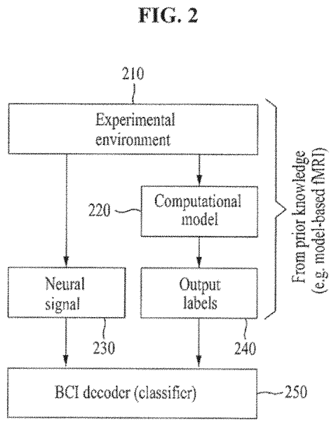

FIG. 2 is a diagram schematically illustrating a BCI system according to an embodiment.

Referring to FIG. 2, a model-based BCI system may be proposed in order to solve a problem in that there is no standard for labeling data for classifying cognitive states. In this method, class output levels 240 may be generated using a separate computational model 220 for a cognitive process. A procedure for the model-based BCI system for classifying cognitive states may be described as follows. In this case, the model-based BCI system may be simply referred to as a BCI system or a meta BCI system.

First, a labeling model may be determined. In model-based fMRI research for examining a target cognitive function of the brain, a proper computational model 220 may be selected. Next, an EEG channel may be selected. An EEG channel close to the location of cortices related to a target cognitive function may be selected. Furthermore, labeling and learning may be performed. A label may be assigned to data using the computational model 220.

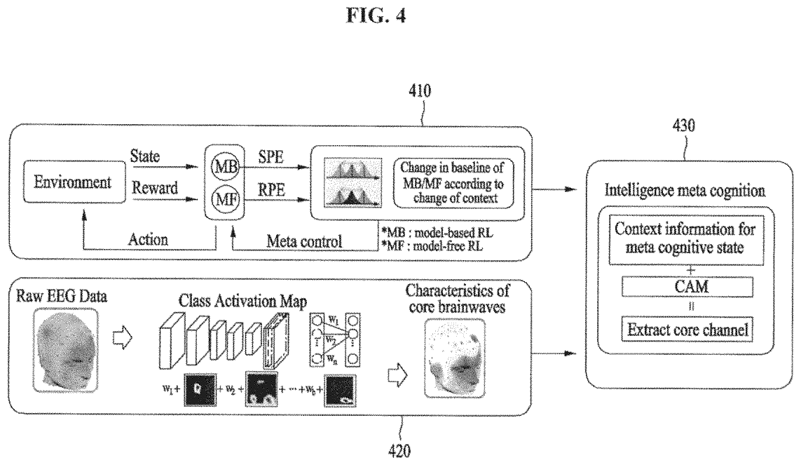

The computational model 220 may be trained by two behavior strategies (e.g., model-based (MB) reinforcement learning (RL) and model-free (MF) RL) based on numerical values from an experimental environment 210. In this case, the experimental environment 210 may be a two-stage Markov decision-making task. The two-stage Markov decision-making task may be used to collect EEG data. The computational model 220 contains a meta control process for continuously modifying that a human being will be using any behavior strategy (or learning strategy). Thereafter, the computational model 220 may provide the output labels 240. In this case, a configuration for providing the output labels 240 through the computational model 220 may be included in a behavior strategy prediction unit 410 of FIG. 4 to be described later. Furthermore, a configuration for extracting a neural signal 230 from the experimental environment 210 may be included in a decision-making prediction unit 420 of FIG. 4.

As described above, in the BCI system, a BCI decoder 250 may be trained by the neural signal 230, extracted from the experimental environment 210, and the output labels 240 determined based on the computational model 220 extracted from the experimental environment 210. The BCI decoder 250 is a classifier for classifying cognitive states, and may include a CNN model.

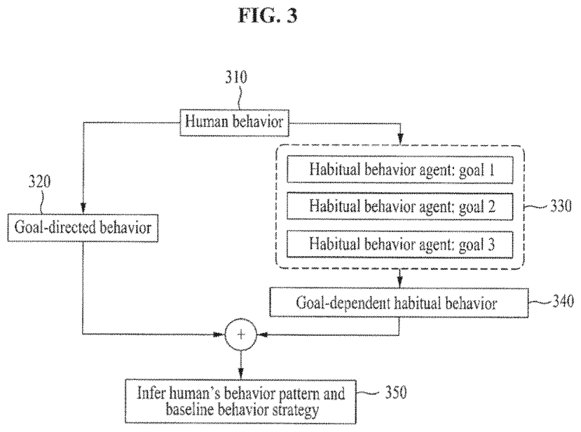

FIG. 3 is a diagram for describing the inference of a behavior strategy and decision making according to an embodiment.

Referring to FIG. 3, the BCI system according to an embodiment may estimate a behavior strategy and decision making included in a human's decision making. In this case, the BCI system may estimate the behavior strategy using a computational model derived from decision-making neuroscience research. Furthermore, the BCI system may estimate the decision making by combining the estimated behavior strategy with characteristics of a brain signal. In this case, if a cognitive state (internal) having a proper classification criterion and said to be a behavior strategy can be estimated, as in the existing BCI system, decision making (external) may also be classified, and a human's decision making and a behavior strategy present in the baseline of the decision making may also be simultaneously estimated.

A human behavior 310 may be divided into a goal-directed behavior 320 and a habitual behavior 330. A human's behavior pattern and a behavior strategy present in the baseline of the behavior pattern may be inferred (350) through the goal-directed behavior 320 and a goal-dependent habitual behavior 340 according to the habitual behavior 330.

A BCI method of estimating a human's decision-making strategy and a behavior pattern based on the decision-making strategy according to an embodiment may include step A of estimating a cognitive state for a behavior strategy inherent in the decision making of a human behavior using a computational model derived from decision-making neuroscience research. Furthermore, the BCI method may further include step B of estimating a cognitive state for the decision making by combining the estimated behavior strategy with characteristics of a brain signal.

Hereinafter, the BCI method is more specifically described using a BCI system for estimating a human's decision-making strategy and a behavior pattern based on the decision-making strategy (simply called a BCI system) according to an embodiment.

FIG. 4 is a diagram for describing a BCI system according to an embodiment.

Referring to FIG. 4, the BCI system according to an embodiment may include the behavior strategy prediction unit 410 and the decision-making prediction unit 420. In some embodiments, the BCI system may further include a learning state estimation model 430.

At step A, the behavior strategy prediction unit 410 may estimate a cognitive state for a behavior strategy inherent in the decision making of a human behavior using a computational model derived from decision-making neuroscience research. In this case, the behavior strategy prediction unit 410 may infer the behavior strategy of a user from brainwaves (or electroencephalography) or fMRI.

The computational model provides a criterion for classifying cognitive state EEG data using neuroscience research. In common neuroscience research, a computational model for each cognitive state is generated, and fMRI data is analyzed. Accordingly, a criterion for classifying cognitive state EEG data may be established using the results of the existing fMRI neuroscience research and a computational model used for the research.

The behavior strategy prediction unit 410 may train the computational model using goal-directed and habitual cognitive states based on numerical values obtained from an experimental environment. Furthermore, the behavior strategy prediction unit 410 may perform meta control for modifying the computational model in response to a change in the numerical value that provides a label for a cognitive state. Thereafter, the computational model of the behavior strategy prediction unit 410 may provide output labels, that is, numerical values of cognitive states.

At step B, the decision-making prediction unit 420 may estimate a cognitive state for the decision making by combining the estimated behavior strategy with characteristics of a brain signal.

The decision-making prediction unit 420 may estimate the cognitive state for the decision making by classifying decision-making strategies using a convolution neural network (CNN). Furthermore, the decision-making prediction unit 420 may estimate the cognitive state for the decision making by visualizing characteristics of the brain signal, associated with each decision-making strategy, in the form of a class activation map (CAM).

Furthermore, the decision-making prediction unit 420 may receive the results of the behavior strategy prediction unit 410 and the decision-making prediction unit 420 through the learning state estimation model 430, and may extract a core channel.

The behavior strategy prediction unit 410 may estimate a cognitive state for a behavior strategy through the following computational model, and may provide output labels, that is, numerical values of the cognitive state.

A reinforcement learning (RL) problem may be represented as a process of finding a behavior strategy that maximizes an expectation (or minimizes costs) for a future reward to be received by an agent. In this case, the behavior strategy is represented as a value of a state or an action in each state. In general, the value is defined as an expectation for a total sum of future rewards to be received by an agent. In a Markov decision process (MDP) problem, the expectation of a reward uses a sample obtained from experiences in which an agent interacts with an environment. The reason why this problem is difficult is that a reward for input (agent's behavior)-output (feedback from an environment) in each state is sparsely given. A reward signal is given in the middle of the interaction in which the input-output is repeated or at the last of the interaction. Furthermore, the reward is not dependent on only a state at timing at which the reward is given, but is dependent on a series of input and output episodes occurred in the past.

In the setting of the MDP, the input and output of each state are represented in a form dependent on an input and output pair in a previous state. If a state shift matrix (P) indicative of information of feedback/reward values (R) for all states (S) and a probabilistic relation between states is given, an optimum strategy may be represented as a value matrix of the states as follows.

v=R+γPv

⇒v=(1−γP)⁻¹R. (1)

However, the above solution may not be applied to a common state for the following reason. A first problem is that P, that is, perfect information of an environment, is not given. If sampling is performed while exploring within the environment during an infinite time, the state shift matrix (P) may be estimated. However, it is difficult to obtain all feedback/reward values (R) for a complicated problem because the above equation can be applied to only a discrete space having a small size. In the aforementioned several problems of common optimum control, the number of states to be considered is too many. In contrast, it is practically difficult to obtain the above matrix itself because an opportunity for sampling is relatively small.

The principle of optimality provides a theoretical base for solving the problem. Assuming that an optimum strategy M* that connects the first state (S0) and a state (Sn) to which the final reward is given is present, an optimum solution Mn−1* that connects a state (Sn−1) and a state Sn is a subset of the optimum strategy M*. Mi−1* is subset of a strategy Mi*. This is described in detail as follows. If a partial strategy at timing at which a reward is obtained is recursively extended, it may be said that the partial strategy becomes the entire strategy that starts from a given state (Si). As a result, it may be said that information on a reward that is finally obtained may be reversely propagated as state-behavior-the feedback sets placed on the past episode until the reward is obtained. Such an ideal conclusion is to be guaranteed under a specific assumption.

A Bellman equation represents a value of a state or behavior, sampled from a strategy, as an expectation of a total sum of rewards using the above principle. A Bellman optimality equation means a relation between a value of an optimum strategy and an expected value. An expectation may be explained in detail in a recursive form using characteristics of the MDP. The above equation may be represented as an update equation of a simple form in which a value (Q(s′,a′)) of a behavior (a′) for a state (s′) to be continued next is incorporated into a behavior value (Q(s,a)) for a current state (S).

Q*(s,a)=E_{(s,a,s′)}[R+γ max_{a′}Q*(s′,a′)]. (2)

In this case, attention needs to be paid to two characteristics. One characteristic is to select a maximum value (max) of behavior values in a next state (s′) and incorporate the selected value into updates. This means that the strategy of an agent itself is assumed to be optimum (i.e., optimistic). The other characteristic is that a model for an environment, that is, a probability distribution for (s,a,s′), is necessary to estimate an expected value.

A cognitive state, that is, a state in which a “behavior strategy” has been determined as a learning strategy, is more specifically described as an example. In this case, a cognitive state is described as an example of a learning strategy, but the present disclosure is not limited thereto and may be applied to all common cognitive states.

A human's learning strategy is represented as RL, and may be divided into MB RL and MF RL. MB and MF are behaviorally related to a goal-directed/habitual learning strategy. In this case, MB/MF corresponds to the name of the learning strategy, and may mean a goal-directed/habitual learning strategy, for example.

The behavior strategy prediction unit 410 may extract context information of a brain level through the computational model. The computational model may be trained by the two learning strategies (i.e., MB/ME) based on numerical values obtained from the experimental environment. The computational model contains a meta control process for continuously modifying that a human being will be using any learning strategy based on the computational model.

Context information for a meta cognitive state may be provided through the behavior strategy prediction unit 410. That is, the computational model may provide output labels.

The decision-making prediction unit 420 may extract brainwave characteristics dependent on the context. As it is said that the context information is a numerical value that provides a label for a cognitive state, a brainwave signal that characteristically appears in each learning strategy may be found.

Accordingly, the learning state estimation module 420 may be aware that which learning strategy is used (i.e., context information) in a meta cognitive state (i.e., a state in which a dominant learning strategy continues to be changed in the two learning strategies), and may characterize a brainwave channel essentially used for each learning strategy using a technical algorithm (i.e., a class activation map (CAM)) for finding a brainwave signal unique to the learning strategy. In this case, the CAM may include a brainwave frequency band and timing in addition to a core brainwave channel. The CAM is important because at which time zone a behavior strategy is changed when the behavior strategy is actually by meta control and where a movement of information is directed through which frequency (i.e., brainwave channel) can be seen.

A BCI decoder for cognitive state estimation and a neural profile extraction technology are described below.

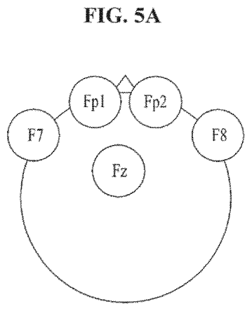

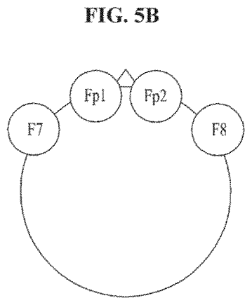

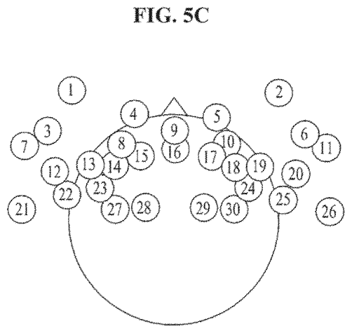

FIG. 5A is a diagram illustrating electroencephalogram (EEG) channel information of a 1-D convolution neural network (CNN) model according to an embodiment. FIG. 5B is a diagram illustrating EEG channel information of a 2-D CNN model according to an embodiment. Furthermore, FIG. 5C is a diagram illustrating EEG channel information of a 3-D CNN model according to an embodiment. That is, FIGS. 5A to 5C illustrate channel information used in the BCI decoder (1-D/2-D/3-D CNN model) according to an embodiment.

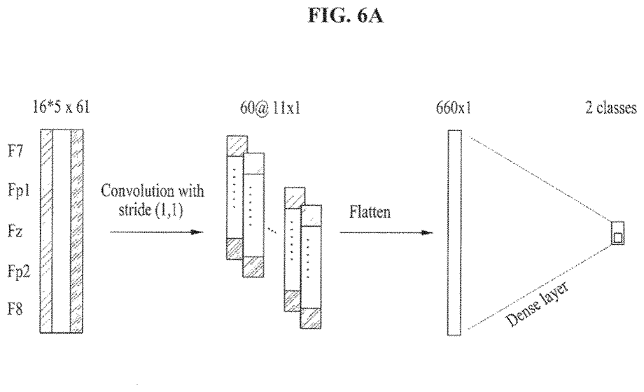

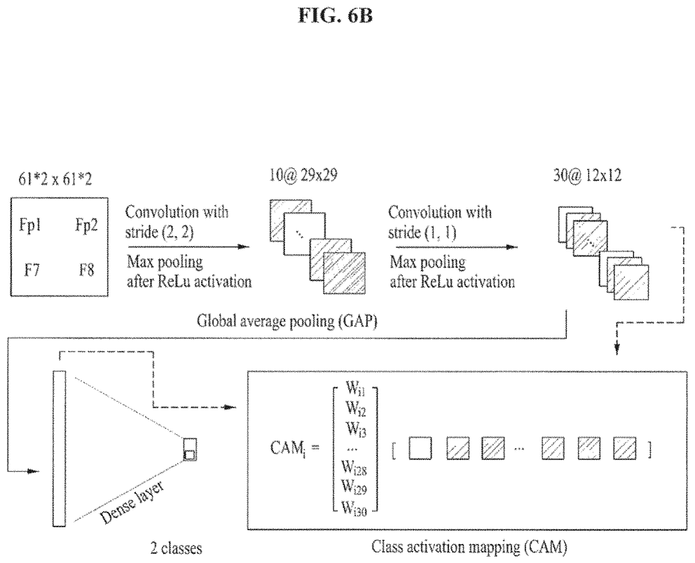

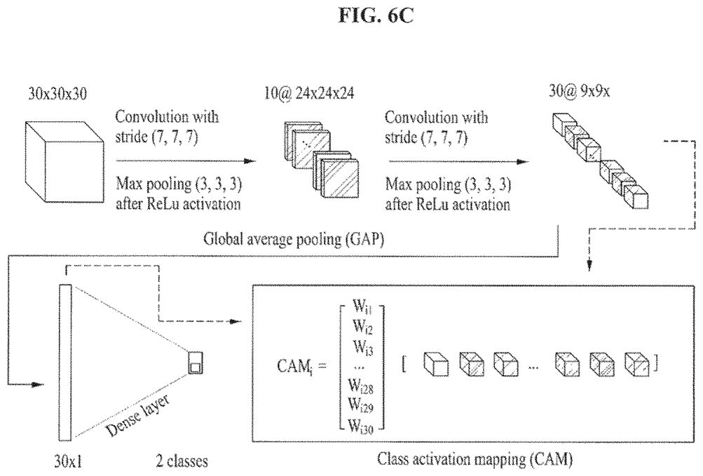

FIG. 6A is a diagram illustrating the structure of the 1-D CNN model according to an embodiment. FIG. 6A illustrates the structure of the 1-D CNN model. The 1-D CNN model has been trained in the form of a connected characteristic matrix. FIG. 6B is a diagram illustrating the structure of the 2-D CNN model according to an embodiment. FIG. 6C is a diagram illustrating the structure of the 3-D CNN model according to an embodiment. The BCI decoder according to an embodiment may be represented in the form of the 1-D/2-D/3-D CNN model. In particular, as in the structures of the 2-D CNN model and the 3-D CNN model, the BCI decoder may be represented as a combination of a CNN and a CAM.

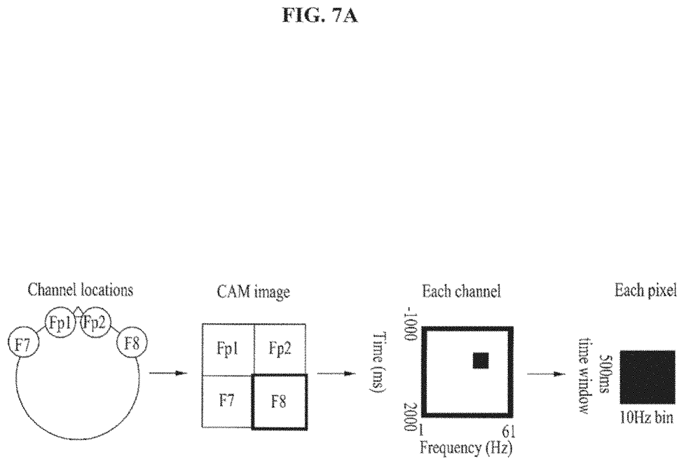

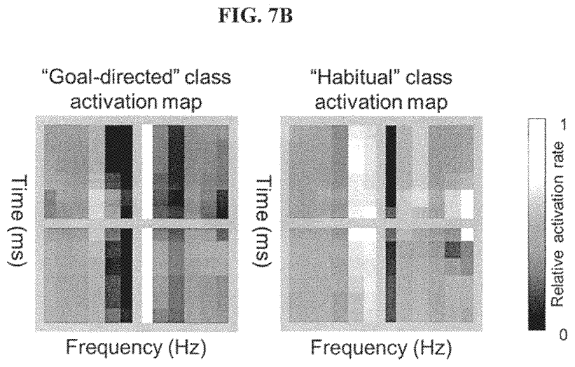

FIG. 7A is a diagram for describing a 2-D CNN class activation map (CAM) according to an embodiment. FIG. 7A shows an actual CAM in the goal-directed and habitual behaviors of a model-based BCI using the 2-D CNN.

FIG. 7B is a diagram illustrating simulation results of the 2-D CNN CAM according to an embodiment. FIG. 7B shows how meaningful information is included in a relative activation rate through colors.

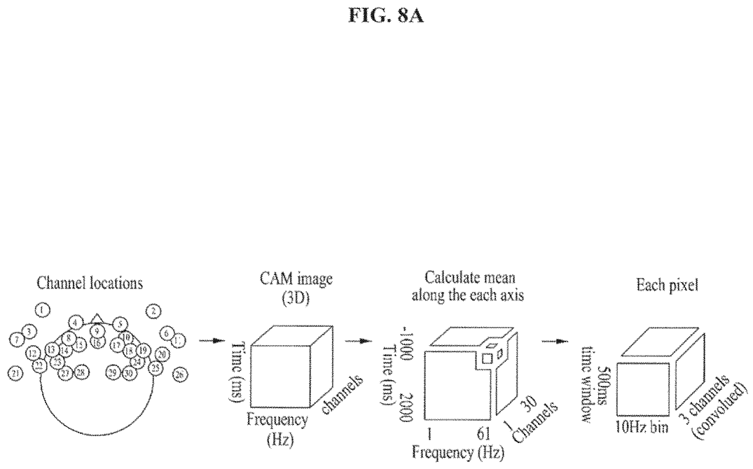

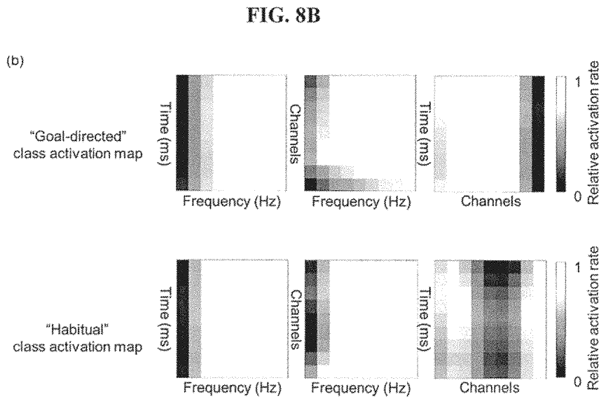

FIG. 8A is a diagram for describing a 3-D CNN CAM according to an embodiment. FIG. 8A shows an actual CAM in the goal-directed and habitual behaviors of a model-based BCI using the 3-D CNN.

FIG. 8B is a diagram illustrating simulation results of the 3-D CNN CAM according to an embodiment. FIG. 8B shows how meaningful information is included in a relative activation rate through colors. As described above, a cognitive state can be visualized using the 3-D CNN CAM and may be extended to an online BCI system.

Table 1 shows the results of a comparison between cognitive state estimation performance of a classifier between the existing system and the model-based BCI system according to an embodiment of the present disclosure. In this case, the classifier may mean a BCI decoder.

TABLE 1

| Classifier | SVM | SVM* | 1D CNN | 2D CNN | 3D CNN |
|---|---|---|---|---|---|
| Accuracy (%) | 69.24 ± | 85.22 ± | 98.73 ± | 95.05 ± | 98.49 ± |
| | 2.99 | 2.03 | 0.86 | 3.09 | 1.45 |

SVM*: data labeled with the computational model

Referring to Table 1, 5 different BCI decoders (in this case, indicated as classifiers) are used. The conventional BCI system and the model-based BCI system according to embodiments of the present disclosure were compared based on the different BCI decoders. As a result of the comparison, it can be seen that an ultra-high performance cognitive state estimator can be designed using the model-based BCI system according to embodiments of the present disclosure.

A support vector machine (SVM) illustrates a case where it is trained by the conventional BCI system. That is, this corresponds to a case where a human being manually determines a label. Furthermore, the SVM* and the 1-D/2-D/3-D CNNs illustrate cases they are trained by the model-based BCI system according to embodiments of the present disclosure. That is, the SVM* and the 1-D/2-D/3-D CNNs illustrate cases a cognitive state estimated by the computational model of the behavior strategy prediction unit is used as a label.

The SVM and the SVM* are very simple, and use the same behavior strategy prediction algorithm called an SVM. In the SVM, a human being manually determines a label. In contrast, the SVM* shows results having more improved performance because it uses the model-based BCI system using the computational model. In other words, the SVM* uses a label estimated by the computational model of the behavior strategy prediction unit and shows rapidly improved performance.

It is expected that the 1-D/2-D/3-D CNNs will have better performance than the previous two SVM models because they are common deep learning models. In this case, the 1-D CNN is a model for rapid prediction because it uses less dimensional data, but for this reason, a CAM cannot be applied to the 1-D CNN. The 2-D/3-D CNNs can apply a CAM and also show high performance. Accordingly, a brainwave channel essentially used for each learning strategy can be characterized through the behavior strategy prediction and the CAM.

Embodiments provide the BCI system based on the results of prior research of a high-dimensional decision-making strategy present in the baseline of a human's decision making behavior. The BCI system may be variously applied as human life aids for inferring a user's decision-making strategy from brainwaves (or electroencephalography), fMRI, etc. As a representative example, an artificial intelligence (AI) secretary using the BCI system may construct a human-centric and user-customized AI system by providing information missed by a user. It is very important to read a state of a user and to selectively provide information important for the user in an Internet of things (IoT) environment. This may also be extended to an advertising proposal system. Furthermore, the inference of a human's decision-making strategy and a behavior pattern based on the decision-making strategy is a key to human-friendly AI and technology development as in the affective computing field. Pieces of human aid AI now being developed merely increase the number of kinds of a possible technology regardless of a human's state. However, AI that understands a human being can be developed through the present disclosure. Furthermore, integration with other conventional BCI technologies in addition to a portion for inferring a human's decision-making strategy may improve the entire human life.

The aforementioned BCI system may include a high-level cognitive state decoder. The high-level cognitive state decoder and a universal cognitive state decoder based on a brain signal using the same are described below. A method and apparatus for predicting an ultra-high performance complex behavior based on the universal cognitive state decoder is more specifically described below.

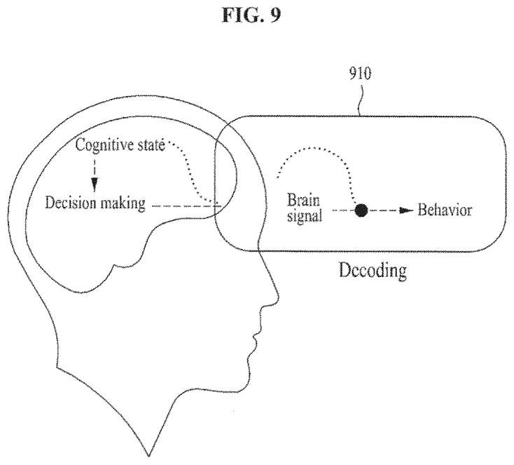

FIG. 9 is a diagram illustrating a complex behavior decoding concept diagram using a high-level cognitive state according to an embodiment.

Referring to FIG. 9, a human's cognitive state is a signal present in the baseline of a behavior, and has been known as an element that produces the complexity of a behavior pattern. In general, various types of human's cognitive states are contextually similar. Accordingly, a task-independent “high-level cognitive state” is present, and may be noninvasively decoded (910) from a frontal lobe. For example, the location of the frontal lobe for the decoding of the high-level cognitive state is ateroversion cortices and frontalis cortices. A decoder for the “high-level cognitive state” may be used to distinguish between cognitive states that now directly/indirectly affects the execution of a task regardless of the type of task, and has generality.

The following embodiments may provide a universal cognitive state decoder through such characteristics, and may predict a human's complicated behavior pattern using the universal cognitive state decoder. According to embodiments, a cognitive state and the selection of a behavior according to the cognitive state may be read with accuracy of about 98%. This may be considered as being the best performance level in the world, which is about 38% higher than that of the existing deep learning-based decoder.

Hereinafter, a universal cognitive state decoding technology in which a neural signal, such as brainwaves, is received as an input based on a functional characteristic of a cognitive state and a technology for implementing a complicated behavior prediction system using the same are described. In such a neural signal decoding technology, neuroscience research for specifying a brain portion related to the estimation of a core cognitive state and engineering research for an efficient decoder design using a deep learning technology have been closely combined. A similar research case is not present in a conventional technology.

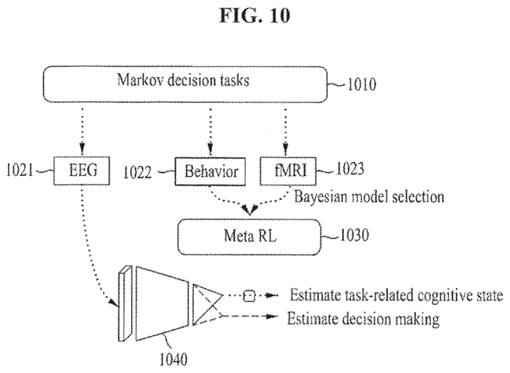

FIG. 10 is a diagram for describing a high-level cognitive state decoder and the design of a behavior signal decoder using the same according to embodiments.

Referring to FIG. 10, a neuroscience-AI convergence type decoding technology for the BCI system is provided. In particular, FIG. 10 illustrates the training of a brain decoder dependent on a frontal lobe-basal ganglia meta reinforcement learning (RL) process.

The high-level cognitive state decoder may estimate a cognitive state using a BCI decoder 1040 by performing meta RL 1030 on a behavior 1022 and fMRI 1123 extracted from a Markov decision-making task 1010. Furthermore, the high-level cognitive state decoder may estimate a user's decision making from EEG 1121, extracted from the Markov decision-making task 1010, using the BCI decoder 1040. In this case, the BCI decoder 1040 may include a CNN and/or an LSTM.

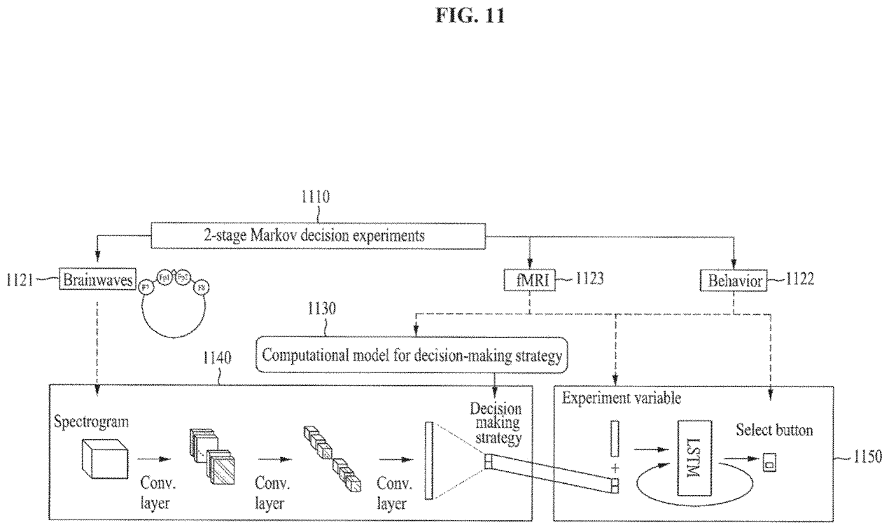

FIG. 11 illustrates a high-level cognitive state decoder and a behavior signal decoding method using the same according to embodiments.

The high-level cognitive state decoder of FIG. 11 is an embodiment of the high-level cognitive state decoder described with reference to FIG. 10. FIG. 11 illustrates a model-based BCI system for reading a decision-making strategy (or behavior strategy) and a selection signal.

A computational model 1130 for a decision-making strategy may assign a label to a decision-making strategy of EEG date in model-based fMRI research. In this case, the decision-making strategy may mean a behavior strategy.

A CNN-based decision-making strategy prediction decoder 1140 has spectrogram as an input extracted from brainwaves (EEG) 1121. The decision-making strategy prediction decoder 1140 may predict a user's decision-making strategy based on the brainwaves 1121 or fMRI 1123 extracted from a Markov decision-making task 1110.

A behavior prediction decoder 1150 based on a long short term memory (LSTM) may use, as an input, an explicit behavior clue (e.g., a state and a goal) and the decision-making strategy decoded by the decision-making strategy prediction decoder 1140. The behavior prediction decoder 1150 may estimate a cognitive state for the decision making by combining the estimated decision-making strategy with characteristics of a brain signal, such as the fMRI 1123 and a behavior 1122. That is, the behavior prediction decoder 1150 may predict what decision making the user will take (e.g., which button the user will press).

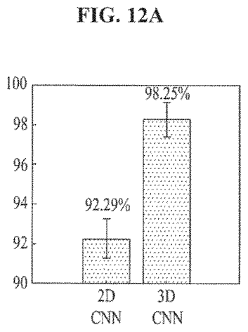

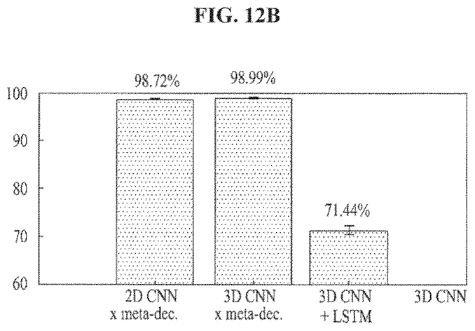

FIG. 12A is a diagram illustrating performance of a decision-making strategy prediction decoder according to an embodiment. FIG. 12A illustrates performance evaluation of the high-level cognitive state decoder and the behavior signal decoder using the same, and illustrates the results of performance evaluation of the decision-making strategy prediction decoders based on the 2-D CNN and the 3-D CNN.

FIG. 12B is a diagram illustrating performance of a behavior prediction decoder according to an embodiment. FIG. 12B illustrates performance evaluation of the high-level cognitive state decoder and the behavior signal decoder using the same. It may be seen that the decision-making strategy prediction decoders based on the 2-D/3-D CNNs and the meta decoder have better performance than those based on the 3-D CNN, and the LSTM and the 3-D CNN.

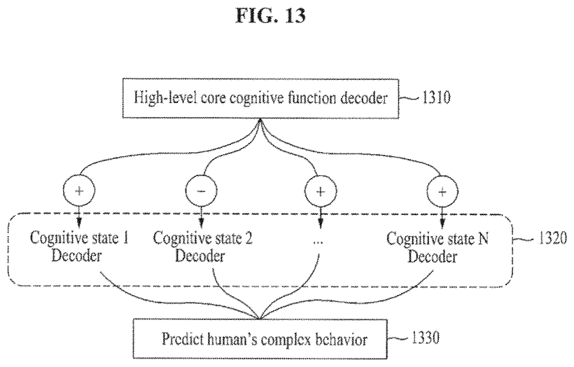

FIG. 13 is a diagram for describing the design of a universal cognitive state decoder using a high-level cognitive state decoder according to an embodiment.

Referring to FIG. 13, the decision-making strategy prediction decoder 1140 described with reference to FIG. 11 has usefulness in decoding a different type of a cognitive state because the decoder decodes a core cognitive state related to decision making, and thus may also predict another complicated behavior. In this case, the decision-making strategy prediction decoder may be referred to as a high-level core cognitive function the decoder 1310.

In other words, a universal cognitive state decoder 1320 capable of decoding a different type of a cognitive state may be configured using the high-level cognitive state decoder 1310 for decoding a core cognitive state related to decision making. A human's complex behavior may be predicted (1330) using the universal cognitive state decoder 1320.

The universal cognitive state decoder 1320 may be configured in the following sequence. A core cognitive state corresponding to an intersection of various types of cognitive states may be selected. The high-level cognitive state decoder 1310 for classifying the core cognitive states may be configured. The new universal cognitive state decoder 1320 may be configured within a short time (or with zero-training) by including a calculated value of the high-level cognitive state decoder 1310 as an input value to another cognitive state decoder. The universal cognitive state decoder 1320 may be used as a human behavior prediction and behavior aid system.

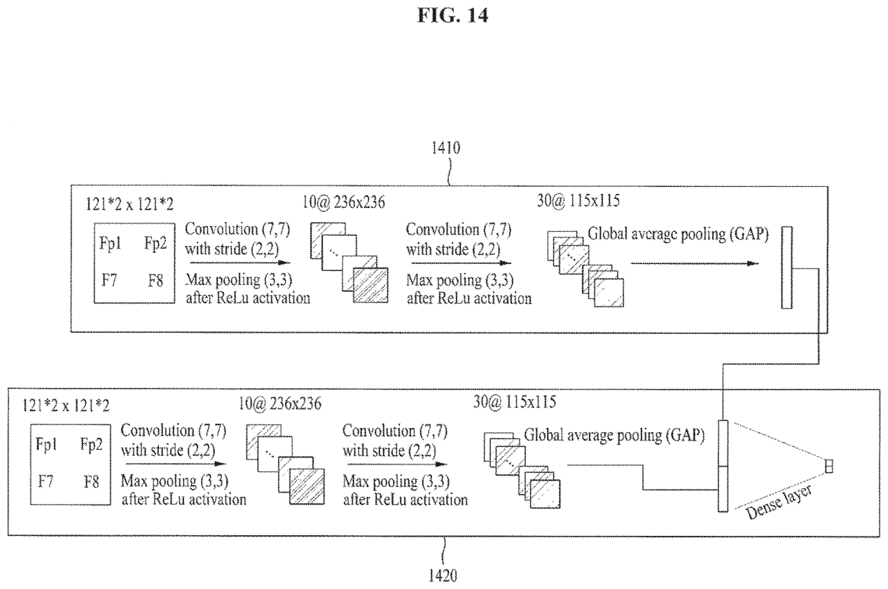

FIG. 14 is a diagram illustrating an apparatus for predicting an ultra-high performance complex behavior using the universal cognitive state decoder based on a brain signal according to an embodiment.

Referring to FIG. 14, a universal complex behavior decoder 1420 may be configured by combining an output value of a high-level cognitive state decoder 1410 with a cognitive state decoder for decoding a different type of a cognitive function (i.e., by a hybrid of the output value and the cognitive state decoder).

More specifically, the apparatus for predicting an ultra-high performance complex behavior using the universal cognitive state decoder based on a brain signal according to an embodiment may include the high-level cognitive state decoder 1410 and the universal cognitive state decoder 1420. In some embodiments, the apparatus for predicting a complex behavior may further include a Markov decision-making task unit. In this case, the high-level cognitive state decoder 1410 may include a behavior strategy prediction unit and a decision-making prediction unit. Furthermore, the universal cognitive state decoder 1420 may also include a behavior strategy prediction unit and a decision-making prediction unit.

First, the Markov decision-making task unit may design a Markov decision-making task for the extraction of a task-independent core cognitive state.

The high-level cognitive state decoder 1410 may classify human's high-level core cognitive states. The high-level cognitive state decoder 1410 may train a high-level cognitive state decoder using a goal-directed cognitive state and a habitual cognitive state, that is, task-independent core cognitive states.

The high-level cognitive state decoder 1410 may include a behavior strategy prediction unit for estimating a core cognitive state for a behavior strategy inherent in the decision making of a human behavior based on a computational model derived from decision-making neuroscience research using a high-level cognitive state decoder. Furthermore, the high-level cognitive state decoder 1410 may include a decision-making prediction unit for estimating a core cognitive state for the decision making by combining the behavior strategy inherent in the decision making of the human behavior with characteristic of a brain signal using the high-level cognitive state decoder. In this case, the decision-making prediction unit may estimate the cognitive state for the decision making by classifying decision-making strategies using a CNN. Furthermore, the decision-making prediction unit may estimate the cognitive state for the decision making by visualizing characteristics of a brain signal, associated with each decision-making strategy, in the form of a CAM.

The universal cognitive state decoder 1420 may predict the human's complex behavior by including a calculated value of the high-level cognitive state decoder in another cognitive state decoder as an input value. The universal cognitive state decoder 1420 may be configured to include the calculated value of the high-level cognitive state decoder in a plurality of other cognitive state decoders as an input value. In this case, the high-level cognitive state decoder 1410 and the universal cognitive state decoder 1420 may be configured with CNN-based decoders.

Furthermore, the universal cognitive state decoder 1420 may predict a complex behavior according to an RL strategy, and may infer computer-recognizable behaviors according to vigilance and non-vigilance.

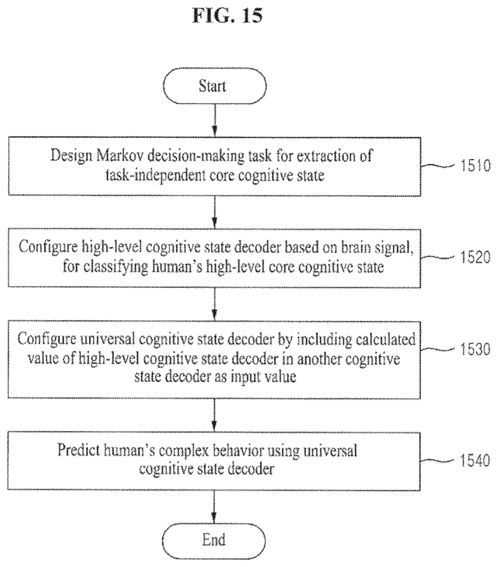

FIG. 15 is a flowchart illustrating a method of predicting an ultra-high performance complex behavior using the universal cognitive state decoder based on a brain signal according to an embodiment.

Referring to FIG. 15, the method of predicting an ultra-high performance complex behavior using the universal cognitive state decoder based on a brain signal according to an embodiment may include step 1520 of configuring a high-level cognitive state decoder based on a brain signal, for classifying a human's high-level core cognitive state, step 1530 of configuring a universal cognitive state decoder by including a calculated value of the high-level cognitive state decoder in another cognitive state decoder as an input value, and step 1540 of predicting the human's complex behavior using the universal cognitive state decoder.

Furthermore, the method may further include step 1510 of designing a Markov decision-making task for the extraction of a task-independent core cognitive state before configuring the high-level cognitive state decoder.

The steps of the method of predicting an ultra-high performance complex behavior using the universal cognitive state decoder based on a brain signal according to an embodiment are described. The method of predicting an ultra-high performance complex behavior using the universal cognitive state decoder based on a brain signal according to an embodiment may be performed by the apparatus for predicting an ultra-high performance complex behavior using the universal cognitive state decoder based on a brain signal according to an embodiment.

At step 1510, the Markov decision-making task unit may design the Markov decision-making task for the extraction of the task-independent core cognitive state.

At step 1520, a high-level cognitive state decoder based on a brain signal, for classifying a human's high-level core cognitive state may be configured. In particular, the high-level cognitive state decoder may be trained using a goal-directed cognitive state and a habitual cognitive state, that is, task-independent core cognitive states.

In this case, a core cognitive state for a behavior strategy inherent in the decision making of a human behavior may be estimated based on a computational model derived from decision-making neuroscience research using the high-level cognitive state decoder. Furthermore, a core cognitive state for the decision making may be estimated by combining the behavior strategy inherent in the decision making of the human behavior with characteristics of a brain signal using the high-level cognitive state decoder. In this case, the cognitive state for the decision making may be estimated by classifying decision-making strategies using a CNN. Furthermore, the cognitive state for the decision making may be estimated by visualizing the characteristics of a brain signal associated with each decision-making strategy in the form of a CAM.

At step 1530, a universal cognitive state decoder may be configured by including a calculated value of the high-level cognitive state decoder in a plurality of other cognitive state decoders as an input value. In this case, the high-level cognitive state decoder and the universal cognitive state decoder may be configured with CNN-based decoders.

At step 1540, a human's complex behavior may be predicted using the universal cognitive state decoder. Furthermore, a complex behavior according to an RL strategy may be predicted using the universal cognitive state decoder, and computer-recognizable behaviors according to vigilance and non-vigilance may be inferred.

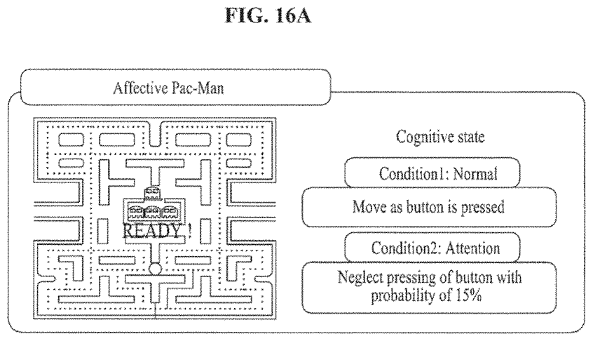

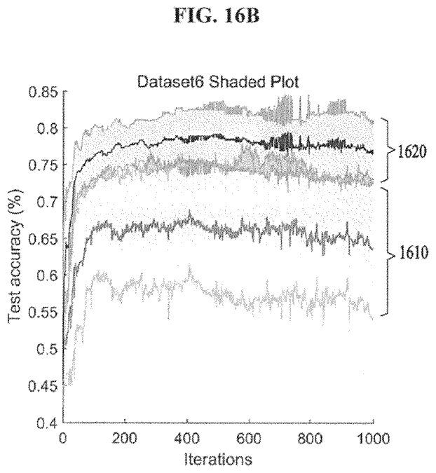

FIG. 16A is a diagram illustrating an example of the universal complex behavior decoder according to an embodiment.

Referring to FIG. 16A, the universal complex behavior decoder according to an embodiment may be applied to the EEG DB of game called Pac-Man. For example, in a normal condition, the universal complex behavior decoder may operate as a user presses a button. In an attention condition, the universal complex behavior decoder may operate regardless of the pressing of a button with a probability of about 15%.

FIG. 16B is a diagram illustrating performance evaluation of the universal cognitive state decoder according to an embodiment.

FIG. 16B illustrates brainwaves measured during the play of the Pac-Man game. As a result of a comparison between a universal cognitive state decoder 1620, that is, a hybrid model, according to an embodiment and an individual cognitive state decoder 1610 for separately decoding a cognitive state in order to classify two types of cognitive states, it can be seen that the universal cognitive state decoder 1620 has much better performance than the individual cognitive state decoder 1610.

According to embodiments, a human's complicated behavior pattern can be predicted by directly reading a cognitive state, that is, an advanced function of the brain, from a passive signal not having a feeling of fatigue in use. Furthermore, embodiments have been specified for generality because various types are inevitably related in the nature of a cognitive function characteristic of the brain.

Embodiments may be applied to a human-robot/computer interaction field. More specifically, since all behaviors of a human being occur based on a high-dimensional cognitive function, embodiments may be applied to all fields in which a human's behavior can be predicted and used. As a representative example, in the affective computing field, an emotion, that is, one of types of human's cognitive states, may be read, and the human's behavior may be assisted based on the read state. Embodiments may assist a human being to achieve excellent performance by constructing a system that efficiently assists the human's behavior through the prediction of different cognitive states (e.g., vigilance and non-vigilance), which are contextually similar to an emotion which may be recognized by a computer, in addition to simply reading the emotion.

Furthermore, in the Internet of things (IoT) field, a cognitive function used to control each device may be various because various devices need to be controlled. In this case, the generality of embodiments is useful because it can assist a human being regardless of a difference between types of cognitive states necessary to control devices and can also be easily translated from another cognitive state decoder if a new device is included in an already constructed IoT ecosystem.

Furthermore, since a core high-level cognitive state is directly related to a human's task execution intelligence, the decoding technology according to embodiments enables job performance profiling for a judge, a doctor, a financial expert, and a military operation commander whose complicated decision making is important. Furthermore, the decoding technology enables prior profiling for a customized system for smart education. Furthermore, the decoding technology can improve the ability to execute a task through the monitoring of the task execution ability.

The inference of a human's cognitive state and a behavior pattern based on the cognitive state may be used as an additional function for the entire human aid system, such as an AI secretary, including the IoT field. Pieces of human aid AI now being developed merely make insensible responses without considering a human's actual cognitive state and situation, and are developed and advanced in a way to simply increase functions. However, the technology according to embodiments which adopts an approach from a viewpoint called the prediction of a cognitive state and behavior based on a human's brainwaves can be developed into a system which understands a human being because the technology can be more friendly to a human being and can provide useful help to a human being through the development of a human-centric technology.

Embodiments may be provided as a core application software and function for a company that develops brainwave measurement equipment and companies that develop healthcare and wearable devices. Embodiments may be used for direct communication with an AI secretary or an IoT system. In addition, embodiments may be used to reduce a danger of an accident of a profession group (e.g., driving or a factory in which a dangerous device is manipulated) whose cognitive state (e.g., vigilance state) is important in an establishment in which it is important to maintain a proper cognitive state. Furthermore, embodiments may also be used for prior profiling for personalized smart education.

The aforementioned apparatus (or device) may be implemented as a hardware component, a software component and/or a combination of them. For example, the apparatus and elements described in the embodiments may be implemented using one or more general-purpose computers or special-purpose computers, for example, a processor, a controller, an arithmetic logic unit (ALU), a digital signal processor, a microcomputer, a field programmable array (FPA), a programmable logic unit (PLU), a microprocessor or any other device capable of executing or responding to an instruction. The processing apparatus (or processor) may perform an operating system (OS) and one or more software applications executed on the OS. Furthermore, the processing apparatus may access, store, manipulate, process and generate data in response to the execution of software. For convenience of understanding, one processing apparatus has been illustrated as being used, but a person having ordinary skill in the art may understand that the processing apparatus may include a plurality of processing elements and/or a plurality of types of processing elements. For example, the processing apparatus may include a plurality of processors or a single processor and a single controller. Furthermore, other processing configurations, such as a parallel processor, are also possible.

Software may include a computer program, code, an instruction or a combination of one or more of them and may configure a processor so that it operates as desired or may instruct processors independently or collectively. The software and/or data may be embodied in any type of a machine, component, physical device, virtual equipment, or computer storage medium or device so as to be interpreted by the processor or to provide an instruction or data to the processor. The software may be distributed to computer systems connected over a network and may be stored or executed in a distributed manner. The software and data may be stored in one or more computer-readable recording media.

The method according to the embodiment may be implemented in the form of a program instruction executable by various computer means and stored in a computer-readable recording medium. The computer-readable recording medium may include a program instruction, a data file, and a data structure alone or in combination. The program instructions stored in the medium may be specially designed and constructed for the present disclosure, or may be known and available to those skilled in the field of computer software. Examples of the computer-readable storage medium include magnetic media such as a hard disk, a floppy disk and a magnetic tape, optical media such as a CD-ROM and a DVD, magneto-optical media such as a floptical disk, and hardware devices specially configured to store and execute program instructions such as a ROM, a RAM, and a flash memory. Examples of the program instructions include not only machine language code that is constructed by a compiler but also high-level language code that can be executed by a computer using an interpreter or the like.

According to embodiments, the universal cognitive state decoder can be provided using the high-level cognitive state decoder based on a brain signal, for classifying a human's high-level core cognitive state. The universal cognitive state decoder based on a brain signal, which can predict a human's complicated behavior pattern using a universal cognitive state decoder and the method and apparatus for predicting an ultra-high performance complex behavior using the same can be provided.

According to embodiments, the universal cognitive state decoder based on a brain signal, which can predict a human's complicated behavior pattern by directly reading a cognitive state, that is, an advanced function of the brain, from a passive signal not having a feeling of fatigue in use, and the method and apparatus for predicting an ultra-high performance complex behavior using the same can be provided.

As described above, although the embodiments have been described in connection with the limited embodiments and drawings, those skilled in the art may modify and change the embodiments in various ways from the description. For example, proper results may be achieved although the above descriptions are performed in order different from that of the described method and/or the aforementioned elements, such as the system, configuration, device, and circuit, are coupled or combined in a form different from that of the described method or replaced or substituted with other elements or equivalents.

Accordingly, other implementations, other embodiments, and equivalents of the claims fall within the scope of the claims.

What is claimed is:

1. A method of predicting a complex behavior, comprising:
configuring a high-level cognitive state decoder based on a brain signal for classifying a human's high-level core cognitive state;
configuring a universal cognitive state decoder by including a calculated value of the high-level cognitive state decoder in another cognitive state decoder as an input value; and
predicting a human's complex behavior using the universal cognitive state decoder.

2. The method of claim 1, further comprising designing a Markov decision-making task for extracting a task-independent core cognitive state, before configuring the high-level cognitive state decoder.

3. The method of claim 1, wherein configuring the high-level cognitive state decoder based on the brain signal for classifying the human's high-level core cognitive state comprises training the high-level cognitive state decoder using a goal-directed cognitive state and a habitual cognitive state which are task-independent core cognitive states.

4. The method of claim 1, wherein configuring the high-level cognitive state decoder based on the brain signal for classifying the human's high-level core cognitive state comprises estimating a core cognitive state for a behavior strategy inherent in decision making of a human behavior based on a computational model derived from decision-making neuroscience research using the high-level cognitive state decoder.

5. The method of claim 1, wherein configuring the high-level cognitive state decoder based on the brain signal for classifying the human's high-level core cognitive state comprises estimating a core cognitive state for decision making by combining a behavior strategy inherent in the decision making of a human behavior with characteristics of a brain signal using the high-level cognitive state decoder.

6. The method of claim 5, wherein estimating the core cognitive state for decision making by combining the behavior strategy inherent in the decision making of the human behavior with characteristics of the brain signal using the high-level cognitive state decoder comprises estimating a cognitive state for the decision making by classifying each decision-making strategy using a convolution neural network (CNN).

7. The method of claim 5, wherein estimating the core cognitive state for decision making by combining the behavior strategy inherent in the decision making of the human behavior with characteristics of the brain signal using the high-level cognitive state decoder comprises estimating a cognitive state for the decision making by visualizing characteristics of a brain signal associated with each decision-making strategy in a class activation map (CAM) form.

8. The method of claim 1, wherein configuring the universal cognitive state decoder by including the calculated value of the high-level cognitive state decoder in the another cognitive state decoder as the input value comprises configuring the universal cognitive state decoder by including the calculated value of the high-level cognitive state decoder in a plurality of other cognitive state decoders as the input value.

9. The method of claim 1, wherein the high-level cognitive state decoder and the universal cognitive state decoder are convolution neural network (CNN)-based decoders.

10. The method of claim 1, wherein predicting the human's complex behavior using the universal cognitive state decoder comprises:
predicting the complex behavior according to a reinforcement learning strategy using the universal cognitive state decoder, and
inferring computer-recognizable behaviors according to vigilance and non-vigilance.

11. An apparatus for predicting a complex behavior, comprising:
a high-level cognitive state decoder based on a brain signal configured to classify a human's high-level core cognitive state; and
a universal cognitive state decoder configured to predict a human's complex behavior by including a calculated value of the high-level cognitive state decoder in another cognitive state decoder as an input value.

12. The apparatus of claim 11, further comprising a Markov decision-making task unit configured to design a Markov decision-making task for extracting a task-independent core cognitive state.

13. The apparatus of claim 11, wherein the high-level cognitive state decoder trains the high-level cognitive state decoder using a goal-directed cognitive state and a habitual cognitive state which are task-independent core cognitive states.

14. The apparatus of claim 11, wherein the high-level cognitive state decoder comprises a behavior strategy prediction unit configured to estimate a core cognitive state for a behavior strategy inherent in decision making of a human behavior based on a computational model derived from decision-making neuroscience research using the high-level cognitive state decoder.

15. The apparatus of claim 11, wherein the high-level cognitive state decoder comprises a decision-making prediction unit configured to estimate a core cognitive state for decision making by combining a behavior strategy inherent in the decision making of a human behavior with characteristics of a brain signal using the high-level cognitive state decoder.

16. The apparatus of claim 15, wherein the decision-making prediction unit estimates a cognitive state for the decision making by classifying each decision-making strategy using a convolution neural network (CNN).

17. The apparatus of claim 15, wherein the decision-making prediction unit estimates a cognitive state for the decision making by visualizing characteristics of a brain signal associated with each decision-making strategy in a class activation map (CAM) form.

18. The apparatus of claim 11, wherein the universal cognitive state decoder is configured to include the calculated value of the high-level cognitive state decoder in a plurality of other cognitive state decoders as the input value.

19. The apparatus of claim 11, wherein the high-level cognitive state decoder and the universal cognitive state decoder are convolution neural network (CNN)-based decoders.

20. The apparatus of claim 11, wherein the universal cognitive state decoder predicts the complex behavior according to a reinforcement learning strategy and infers computer-recognizable behaviors according to vigilance and non-vigilance
